(19) United States
(12) Patent Application Publication
(10) Pub. No.: US 2016/0015839 A1
(43) Pub. Date: Jan. 21, 2016

(54) Rare Earth Doped Nanoparticles for Use in Hyperthermia Treatment of Cells

(71) Applicant: University of Tartu, Tartu (EE)

(72) Inventors: Yury Orlovskiy, Tartu (EE); Alexander Vanetsev, Tartu (EE); Ilmo Sildos, Tartu (EE); Alexandr Popov, Tartu (EE); Viktor Loschenov, Moscow (RU); Ryabova Anastaslya, Moscow (RU); Konstantin Pukhov, Moscow (RU)

(21) Appl. No.: 14/804,533

(22) Filed: Jul. 21, 2015

(30) Foreign Application Priority Data
Jul. 21, 2014 (GB) 1412935.7
Oct. 20, 2014 (GB) 1418541.7

(51) Int. Cl. A61K 51/12

(52) U.S. Cl. CPC A61K 51/1244; A61K 51/1234

(57) ABSTRACT

An approach to hyperthermia for cancer treatment based on multiphonon relaxation of optical excitation in rare-earth (particularly Dy³⁺) doped nanocrystals after laser irradiation allows fast and accurate local heating to a preset temperature. A collection of nanoparticles suitable for use in hyperthermia treatment of cancerous and non-cancerous cells by laser irradiation in the wavelength of the transparency window of biological tissue (800 nm-1300 nm) preferably 800-900 nm is provided, where each nanoparticle comprises a crystalline host structure, and at least one species of rare-earth dopant ion.

### BACKGROUND

The present invention relates to nanoparticles heatable by laser radiation. The invention particularly relates to nanoparticles of rare-earth doped crystalline dielectric or semiconductor material for localised heat treatment and imaging of biological materials, such as malignant (i.e. cancerous) and non-malignant cells.

The most common and effective current methods for cancer treatment include surgery, chemotherapy and ablation. Surgical treatment can be effective if the tumor is localized and operable, which largely depends on the type of cancer and the stage of its development. Apart from limited applicability the main demerit of surgical treatment is removal of large parts of surrounding healthy tissues, which is necessary to ensure that all of the cancer is taken. Chemotherapy is based on the application of drugs, destroying cancer cells at a higher rate than normal cells. All current methods of chemotherapy are non-localized and affect the whole body of the patient. Since this treatment is based on very toxic and bioactive chemicals, side effects usually are extremely severe and may include internal hemorrhage, dysfunction of liver and kidneys, hair loss etc. Chemotherapy is especially dangerous for children with rapidly developing brains. Alternatively, ablation of tumors involves their low temperature (cryotherapy) or high temperature (hyperthermia) treatment destroying cancer cells. The main goal of the ablation is to raise or decrease the local temperature to such extent that cancer cells inevitably die leading to shrinkage or in the best case elimination of the cancer tumor. The location of the tumor must be clearly established beforehand, thus, the preliminary imaging of the tumor is essential. A variety of methods for hyperthermia are used today, including direct localized or delocalized application of heat through thermal blankets, the use of radio-frequency waves to heat up tissues, the use of optical and heat probes inserted in the tumor etc. Authors of the U.S. Pat. No. 6,530,944 suggest using plasmonic effect of gold nanoparticles distributed in the tumor to convert near IR irradiation into heat.

Many nanoparticles with specific physical properties in electrical, magnetic, acoustic, optical or thermal features have been tried to induce various enhanced hyperthermia, aiming to significantly improve the treatment efficiency of conventional heating. The main problem is the proper localization of the hyperthermia treatment, which is almost unachievable with IR or electromagnetic irradiation focusing.

Despite all the advantages of possibly very local and non-invasive treatment, hyperthermia is still not well established clinical tool. The main reasons are massive destruction of non-tumor cells in the case of high intensity of treatment or insufficient efficiency of destruction of localized tumor in the case of lower intensity of treatment. Recently a lot of work with magnetic nanoparticles for targeted heat delivery has emerged. Here, bio-active magnetic nanoparticles, which are concentrated by one means or another in the cancer tumor, are used as emitters of heat. The heating of nanoparticles appears as a result of irradiation with the high frequency electromagnetic field. The main disadvantage of this approach is that though it is possible to localize heat emission, still it is not possible to localize the influence of the EM field. And to achieve reasonable efficiency of heating it is necessary to apply high power EM field, which is rather harmful for the human body, especially in the case of the brain or lung cancer treatment. Also it is worthy to note that these techniques require using of very expensive and sophisticated instrumentation.

Authors of the U.S. Pat. No. 6,530,944 suggest using near IR irradiation to produce plasmonically induced heat from gold nanoparticles distributed in the tumor. This method solves the problem of the localization of the excitation energy however, the efficiency of the suggested process is very low. It follows from the results presented that the maximal temperature achieved after 10 minutes of laser irradiation is only 33° C. that is even less than the temperature of the human body. So, the dose of laser irradiation, which raises the local temperature to 33 C is very high and the temperature is not enough for hypethermia.

### SUMMARY OF THE INVENTION

The invention aims to overcome the above problems by providing a novel approach to hyperthermia for cancer treatment based on multiphonon relaxation of optical excitation in rare-earth (particularly Dy³±) doped nanocrystals after laser irradiation that allows fast and accurate local heating to a preset temperature.

In an embodiment, a collection of nanoparticles suitable for use in hyperthermia treatment of cancerous and non-cancerous cells by light, preferably laser, irradiation in the wavelength of the transparency window of biological tissue (800 nm 1300 nm) preferably 800-900 nm is provided, wherein each nanoparticle comprises a crystalline host structure, and at least one species of rare-earth dopant ion.

The at least one species of rare-earth dopant ion may be selected from the list of Dy³⁺, Pr³⁺, Nd³⁺, Sm³⁺, Eu³⁺, Tb³⁺, Ho³⁺, Er³⁺, and Tm³⁺ ions.

The crystalline host structure may be a dielectric such as a phosphate, a vanadate, a molibdate, a tungstate, an oxide or a fluoride, or a semiconductor material.

The heating effect is observed at low dopant concentrations, for example the concentration of the at least one species of dopant may be in the range to 5 to 100 molar %. However, the heating effect is proportional to the dopant concentration, therefore the higher the dopant concentrations the higher the change in temperature of the nanoparticles upon irradiation, therefore the dopant concentration is preferably in the range 30 to 100 molar %, more preferably in the range 80 to 100 molar %, more preferably in the range 90 to 100 molar %, or still more preferably 95.0 to 100.0 molar %.

The average diameter of the nanoparticles may be in the range 5 to 500 nm, for some cancer cells optimal range is 20 to 60 nm, but this depends on specific cancer cells. The diameter of the nanoparticles may also be in the range 10 to 50 nm, 50 to 70 nm or 100 to 1000 nm.

The heat transfer mechanism is based on direct transformation of the energy of laser excitation to nanocrystals lattice vibrations through the process of multiphonon relaxation of the energy of optical excitation in the rare-earth doped crystals.

The technique allows full and easy control of the local temperature around the sample with 1 (one) degree° C. accuracy and fast heating and cooling in several seconds.

The crystal lattice may be double or triple doped by any combinations of Dy³⁺, Pr³⁺, Nd³⁺, Sm³⁺, Eu³⁺, Tb³⁺, Ho³⁺, Er³⁺, Tm³⁺, and Yb³⁺ ions so that a fluorescence signal is easily detectable when imaging in the transparency window of biological tissues.

In an embodiment, the nanoparticles may have a core of a first host material doped with one or more types of rare earth dopant ions and a shell of a second host material doped with one or more types of rare earth dopant ions. The first and second host material may be the same material, ie one type of host material for the core and the shell, with a core doped with one or more types of ion and a shell doped with one or more different types of ion. The core dopant ions may be of the same type as the shell dopant ions. The concentration of the dopant ions in the core may be less than 1 mol % and are suitable for imaging and the concentration of the dopant ions in the shell is in the range 30-100 mol % and suitable for heating when irradiated with electromagnetic radiation, for example laser light in the infrared, visible or ultraviolet parts of the spectrum.

Imaging, diagnostics, and very high locality of hyperthermia for treatment by one type of crystalline nanoparticles is possible.

The efficiency of the process allows low doses of laser irradiation of human body for treatment.

It is not obvious to provide heating without a metallic shell in the desired spectral range of optical transparency of biological tissues. Conventionally such heating has exploited a plasmonic mechanism whereby a metal shell on the nanoparticle is provided and the thickness of a metal shell is selected to match the desired wavelength. It is known to dope semiconductor nanoparticles with small quantities of rare earth materials but this is for imaging purposes (as opposed to heating) and therefore exploits a radiative relaxation mechanism; large dopant quantities would quench radiative recombination, which disinclines the skilled person to look to the high dopant concentrations of the present invention. In the present invention, a phonon heating mechanism is exploited which requires a much higher rare-earth dopant concentration. The specific dopant concentrations used in the present invention are discussed in more detail below. The optical absorption transition of a rare-earth dopant in a dielectric or semiconductor is selected for a specific wavelength; heating is due to dissipation of optical excitation energy in the nanocrystal matrix through non-radiative multiphonon relaxation.

Rare-earth ions with specific energy levels with desired energies (wavelength) of absorption transition are selected. Those levels have next lower lying energy level with the energy gap allowing from one to three phonon transitions to occur with the rates much higher than the rates of the photon emission transitions from the initially excited level. At the same time the nanocrystal matrix with high maximal phonon frequency is chosen, e.g. YPO₄, to find such an excited level which matches the required conditions for optical energy relaxation. The heating by phonons provides much better locality and rate of heating and cooling than heating by electromagnetic radiation produced by surface plasmon resonance in gold.

In an embodiment, the nanoparticles may be conjugated with molecules that specifically bind to a target cell. Such molecules may be antibodies suitable for the formation of an antigen/antibody complex with the target cell or liposomes having targeting ligands suitable for the formation of a ligand/receptor complex with the target cell.

The nanoparticles may be used in the hyperthermia treatment of over-proliferating cells such as malignant cells.

The nanoparticles may be delivered in the form of a pharmaceutical composition containing the collection of nanoparticles.

In a further embodiment, a method of inducing localised hyperthermia in target cells is provided, the method comprising the steps of delivering nanoparticles of the type as herein described to cells and exposing the nanoparticles to electromagnetic radiation.

In a still further embodiment, a method of inducing localised hyperthermia and imaging target cells is provided, the method comprising the steps of delivering nanoparticles of the type herein to cells and exposing the nanoparticles to electromagnetic radiation and detecting the absorption, fluorescence or scattering of the radiation to simultaneously heat and view the target cells. The electromagnetic radiation used in the above methods may have a wavelength in a biological transparency window of 800-900 nm. The above methods may be applied to cells in vitro. Alternatively, the functionalised nanoparticles of the types described herein may be incorporated in a pharmaceutical composition and administered to a human or an animal.

### BRIEF DESCRIPTION OF THE DRAWINGS

The application contains at least one drawing executed in color. Copies of this application with color drawings will be provided by the US Patent and Trademark Office upon request and payment of the necessary fee.

FIG. 1 is an energy level diagram for YPO₄:Dy³⁺ doped crystalline nanoparticles.

FIG. 2 shows the temperature kinetics of the 1° Dy³⁺:YPO₄ nanoparticles at laser irradiation wavelength of 809 nm.

FIG. 3 shows the temperature kinetics of the 5% Dy³⁺:YPO₄ nanoparticles at laser irradiation wavelength of 809 nm.

FIG. 4 shows the temperature kinetics of the DyPO₄ nanoparticles at laser irradiation wavelength of 809 nm and average power of 60 mW.

FIG. 5 shows the temperature kinetics of the DyPO₄ nanoparticles at laser irradiation wavelength of 811 nm and average power of 15.5 mW.

FIG. 6 shows the Reflectance spectrum of the DyPO₄ sample at room temperature measured by Laser Electronic LESA-01-Biospec spectrum analyzer with tunable femtosecond Chameleon laser, Coherent. The spectra are recorded by diffuse reflection of white light from a thin layer of powder. Y axis is the relative units.

FIG. 7 shows the dependence of the local temperature increase ΔT on the laser power under pulsed laser excitation directly into the ⁶F_{5/2} level of the Dy³⁺ in the DyPO₄ nanocrystals with tₚ=140 fs and f=80 MHz at 811 nm (open circles).

FIG. 8 shows the dependence of the local temperature increase ΔT after laser irradiation versus the concentration of Dy³⁺ in the Y₁₋ₓDyₓPO₄ nanocrystals (solid circles).

FIG. 9 is an energy level diagram with indication of multiphonon transitions (solid arrows downward) in Dy³⁺:YPO₄ crystalline nanoparticles under direct laser excitation into the ⁶F_{3/2}, ⁶F_{5/2}, and ⁶F_{7/2} levels of the Dy³⁺ ion.

FIG. 10 shows the local temperature increase ΔT of the Y₁₋ₓDyₓPO₄ powder taken from the hottest pixel of the image of the powder surface on an IR camera after laser irradiation, versus laser power. Excitation was into the ⁶F_{5/2} (811 nm) and ⁶F_{7/2} (914 nm) levels of Dy³⁺ in the scanning microscope spot mode. Temperature measurements were performed when approaching the steady-state.

FIG. 11 shows the local temperature increase ΔT of the Y₁₋ₓDyₓPO₄ powder taken from the hottest pixel of the image of the powder surface on the IR camera after 100 mW laser irradiation versus the concentration of Dy³⁺. Excitation was done in the scanning microscope spot mode. Temperature measurements were done when approaching the steady-state.

FIG. 12 shows the local temperature kinetics of the hottest area of the DyPO₄ nanocrystals powder (0.425×0.425 mm) taken from the hottest pixel of the image on the IR camera of the powder surface under laser irradiation in the scanning microscope spot mode with the average power of 30 mW at different excitation wavelengths, from top to bottom 811, 914, 760, and 850 nm. The laser was switched, on and off, every second.

FIG. 13 shows XRD patterns of the Y₁₋ₓDyₓPO₄ nanoparticles with x=0.01 (upper) and 1 (lower curve).

FIG. 14 shows a TEM image and particles size distribution of the Y_{0.99}Dy_{0.01}PO₄ nanoparticles.

FIG. 15 shows a TEM image and particles size distribution of the Y_{0.525}Dy_{0.475}PO₄ nanoparticles.

FIG. 16 shows a TEM image and particles size distribution of the DyPO₄ nanoparticles.

FIG. 17 is a diagram showing an experimental set up of how temperature measurements were obtained.

FIG. 18 shows a detailed view of FIG. 2.

FIG. 19 shows a detailed view of FIG. 3.

FIG. 20 shows a detailed view of FIG. 4.

FIG. 21 shows a detailed view of FIG. 5.

### DETAILED DESCRIPTION OF THE PREFERRED EMBODIMENT

The main criterion for the selection of the doping rare-earth ion and the nanocrystalline host is its ability to absorb a laser light at specific wavelength and the ability for heat production depending on the amount of light absorbed, which is equal to the amount of energy of emitted phonons ΔE. In a single frequency model of crystal matrix vibrations it is ΔE=pØω_{eff.}, where p is the number of phonons bridging the energy gap ΔE between an excited energy level and the nearest below, and ω_{eff.} is an effective phonon frequency. Obviously, the higher the frequency the greater the energy gap ΔE between the levels can be bridged with the same amount of phonons, the more thermal energy is instantly passed to the lattice and the stronger nanoparticle heats. In this regard, the crystal matrixes with higher phonon frequencies are better, e.g. oxides, phosphates, molybdates, vanadates, and tungstates. Among these matrices we will select those with the highest specific heat capacity according to the formula

C_{V}(T)ΔT=NΔE, (1)

where C_{V} is the specific thermal capacity (by volume) of the matrix, N—the number of emitters per unit volume, i.e. the concentration of the rare-earth dopant.

An example nanoparticle synthesis method will now be outlined. Water dispersible crystalline nanosized particles of dysprosium orthophosphate or solid solutions of yttrium and dysprosium orthophosphates, undoped or doped with other rare-earth ions (including Pr³⁺, Nd³⁺, Sm³⁺, Eu³⁺, Tb³⁺, Ho³⁺, Er³⁺, Tm³⁺, and Yb³⁺), are prepared by microwave-hydrothermal treatment of phosphate gels, precipitated from aqueous solutions of corresponding metals salts, according to the following steps:

1) Nitrates of dysprosium, yttrium and (possibly) dopant ions taken in stoichiometric ratios are dissolved in deionized water to form a solution, hereafter referred to as Solution 1. The total concentration of metal ions in Solution 1 shouldn't exceed 0.5 M.

2) Potassium or sodium phosphate (basic) is dissolved in deionized water to form a solution, hereafter referred to as Solution 2. The amount of potassium phosphate is equal to the summarized amount of rare-earth nitrates in Solution 1.

3) Solution 1 is added to Solution 2 drop-wise under vigorous stirring to form a precipitate, hereafter referred to as Precipitate 1.

4) Precipitate 1 together with the mother solution is transferred to an autoclave, sealed and exposed to microwave-hydrothermal treatment for 1-4 hours at a temperature in the range of 150-300° C. to form a second precipitate, hereafter referred to as Precipitate 2.

5) Precipitate 2 is removed from the autoclave, washed with deionized water several times and dried at 100° C. for 5-10 hours.

6) Precipitate 2 is re-dispersed in water using ultrasonication to form colloidal solution of crystalline nanosized particles of dysprosium orthophosphate or solid solutions of yttrium and dysprosium orthophosphates, un-doped or doped with other rare-earth ions.

The concentration of dopant in the nanoparticle is controlled by the ratio between rare-earth nitrates during preparation of Solution 1. The ratio between Solution 1 and 2 affects the cation:anion composition of the salt, for example, for phosphates it is always 1, as the formula of any rare-earth phosphate is RPO₄ (1:1).

Alternatively, sulphates or chlorides of dysprosium, yttrium and optionally dopant ions can be used instead of nitrates during step 1.

As a further alternative, potassium or sodium fluoride or molibdate or vanadate or tungstate, etc. can be used instead of potassium or sodium phosphate to form Solution 2 in order to synthesize nanoparticles of corresponding rare-earth fluorides, molibdates, vanadates, tungstates or other dielectric or semiconductor material.

A nontoxic surfactant, such as Proxanol-268 (registered trademark) which is a poloxamer-type polymer, or sodium citrate, poly(methylmethacrylate), polyvinylalcohol, polyethyleneglycol or others may be added to Solution 1 to enhance the dispersability of Precipitate 2. The amount of surfactant used is equal to the summarized amount of rare-earth salts in Solution 1.

Hydrothermal treatment with conventional heating can be used instead of microwave-hydrothermal treatment.

A mixture of water with a high boiling organic solvent such as alcohol, glycol, amine, amide, acid, complex ether, ketone, etc can be used instead of water for preparation of Solutions 1 and 2. This has the effect of decreasing the size of the final nanoparticles. The water to solvent ratio may be chosen in the range from 0 to 1. Using only water as a solvent, nanoparticles of 30-500 nm size are obtained, depending on conditions of synthesis and type of anion.

As an example, for a mixture of water and ethylene glycol in a 1:1 ratio, the size of rare earth-doped yttrium phosphate nanoparticles is around 20 nm, comparing to 40-60 nm for a pure water. For pure oleylamine as a solvent to produce rare earth-doped gadolinium or yttrium oxides, the obtained nanoparticles size is 5-10 nm.

The list of dielectric and semiconductor host crystal materials is only an example, and more can be included. Any dielectric or semiconductor material is potentially suitable as a host material, but materials with low conductivity are preferred, as high conductivity prevents luminescence, and may prevent thermal emission. The common feature is that they should form compounds with rare-earths, which 1) luminescent, 2) can be obtained in the form of nanoparticles.

After production of suitable nanoparticles their response to irradiation should be tested.

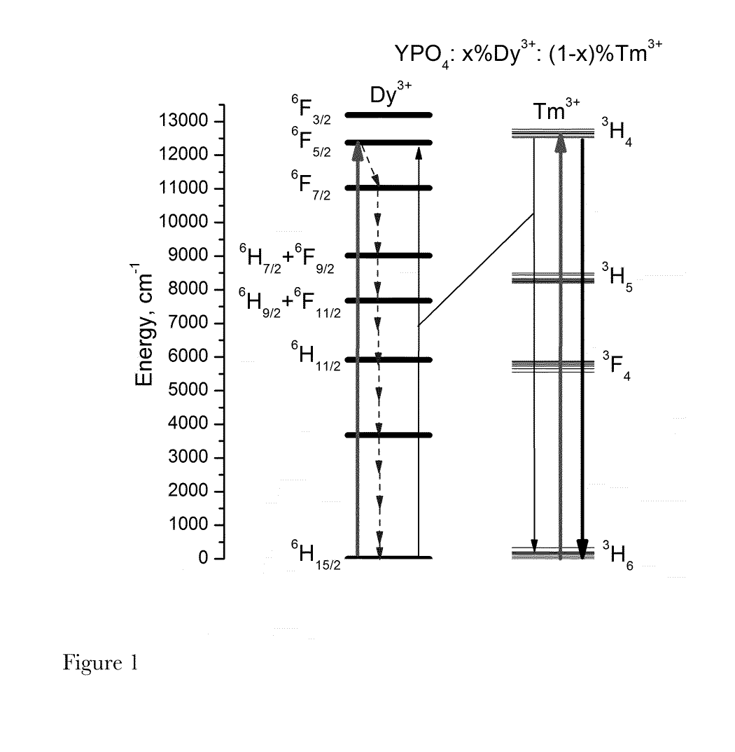

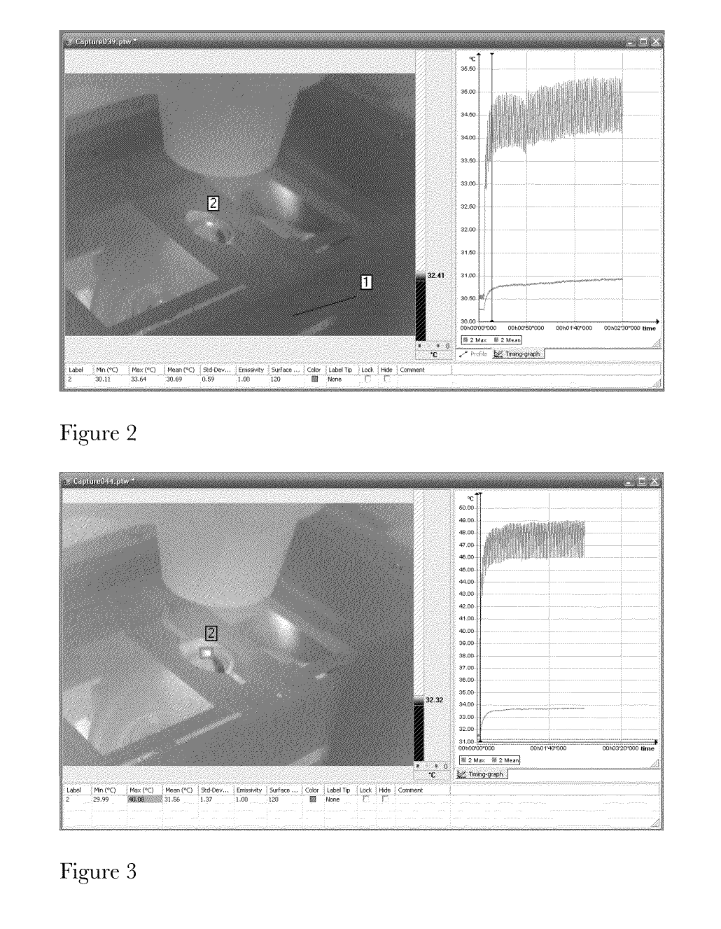

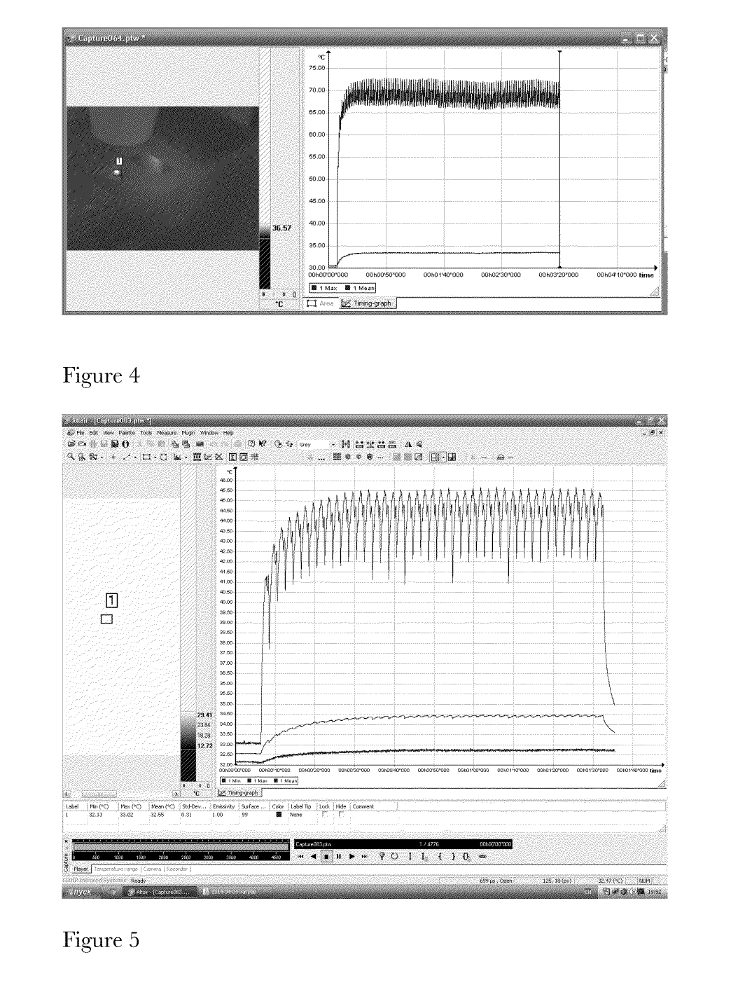

As an example, using method described above, YPO₄ nanocrystalline particles were synthesised of 60 nm average size doped by different concentrations of the Dy³⁺ ion (1, 5, and 100%) and excite them directly into the ⁶F_{5/2} level (FIG. 1) by pulsed tunable laser with 140 fs pulse duration, repetition frequency 80 MHz, and average power of 60-140 mW at 809 nm wavelength that is into the peak of the spectral line of the 6H15/2-6F5/2 optical transition lies in the transparency window of biological tissues 800-900 nm (FIG. 2). We measure the temperature of nanoparticles by Remote High Sensitive IR camera JADE MWIR SC7300M, Cedip. The local temperature of the nanoparticles in place of laser irradiation rises with increase of the concentration of Dy³⁺ comparing to the average temperature of the sample. The temperature increase is 5.5° C. for 1% of Dy³⁺ (FIG. 3), 16.5° C. for 5% of Dy³⁺ (FIG. 4) at 141 mW of excitation laser power, and 40° C. for 100% of Dy³⁺ (FIG. 5) even at lower (60 mW) excitation of laser power. This evidently indicates that the process of multiphonon relaxation inside rare-earth dopant is responsible for the heating.

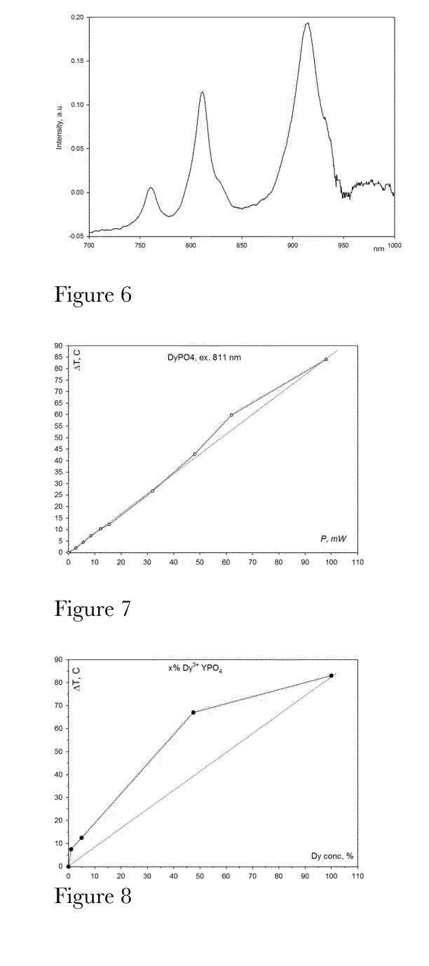

At the same time the response time of the system is very fast. For example, the rise time of the sample temperature from 32.5 degree ° C. to 45.0 degree ° C. is 13 seconds only and the decay time back to 32.5 degree ° C. is approximately 8 seconds (FIG. 6). This indicates very high locality of heating that is very important for selective treatment of cancer tumors without disturbing healthy tissues. The decrease from the treatment temperature 45.5 degree° C. to the pretreatment temperature 41 degree° C. is within one second only that evidently shows that surrounding medium is practically not heated. In an embodiment and as shown in the FIG.s, the laser was switched off every one second and switched on again for one second to check the rate of heating and cooling; if the laser was kept constantly on, the temperature and continuous heating would be even higher. And this is a real advantage over the U.S. Pat. No. 6,530,944 patent. Moreover, the laser power used for heating is low enough, 15.5 mW only that together with fast heating time means extremely low doses of laser irradiation of human body for treatment.

FIG. 1 is an energy level diagram of multiphonon relaxation (dashed arrows) in the YPO₄:Dy³⁺ doped crystalline nanoparticles under direct laser excitation into the ⁶F_{5/2} level of the Dy³⁺ ion (left grey arrow) or after sensitization of multiphonon relaxation as a result of nonradiative energy transfer from the Tm³⁺ ion (black arrows) excited into the ³H₄ level at the ³H₆-³H₄ transition with high absorption cross-section (right grey arrow). The excitation wavelength lies in a first transparency window of biological tissues 800-900 nm. The emission wavelength at the ³H₄-³H₆ transition of Tm³⁺ is also within the transparency window.

FIG. 2 shows the absorpton spectrum of the DyPO₄ sample at room temperature measured by Laser Electronic LESA-01-Biospec spectrum analyzer with tunable femtosecond Chameleon laser, Coherent. The absorption spectra are recorded by diffuse reflection of white light from a thin layer of powder. Y axis is the relative units.

FIG. 3 shows the temperature kinetics of the 1% Dy³⁺:YPO₄ nanoparticles at pulsed laser irradiation with 140 fs pulse duration, repetition frequency 80 MHz, and average power of 141 mW at 809 nm wavelength measured by Remote High Sensitive IR camera JADE MWIR SC7300M, Cedip. The laser is switched off every one second and switched on again for one second to check the rate of heating and cooling. The upper curve is a maximal temperature in the place of laser irradiation and the lower curve is the average temperature of the sample during irradiation.

FIG. 4 shows the temperature kinetics of the 5% Dy³⁺:YPO₄ nanoparticles at pulsed laser irradiation with 140 fs pulse duration, repetition frequency 80 MHz, and average power of 141 mW at 809 nm wavelength measured by Remote High Sensitive IR camera JADE MWIR SC7300M, Cedip. The laser is switched off every one second and switched on again for one second to check the rate of heating and cooling. The upper curve is a maximal temperature in the place of laser irradiation and the lower curve is the average temperature of the sample during irradiation.

FIG. 5 shows the temperature kinetics of the DyPO₄ nanoparticles at pulsed laser irradiation with 140 fs pulse duration, repetition frequency 80 MHz, and average power of 60 mW at 809 nm wavelength measured by Remote High Sensitive IR camera JADE MWIR SC7300M, Cedip. The laser is switched off every one second and switched on again for one second to check the rate of heating and cooling. The upper curve is a maximal temperature in the place of laser irradiation and the lower curve is the average temperature of the sample during irradiation.

FIG. 6 shows the temperature kinetics of the DyPO₄ nanoparticles at pulsed laser irradiation with 140 fs pulse duration, repetition frequency 80 MHz, and average power of 15.5 mW at 811 nm wavelength measured by Remote High Sensitive IR camera JADE MWIR SC7300M, Cedip. The laser is switched off every one second and switched on again for one second to check the rate of heating and cooling. The upper curve is a maximal temperature in the place of laser irradiation, the middle curve is an average temperature, and the lower curve is the minimal temperature of the sample during irradiation.

It should be noted that the pulsed irradiation of the sample is to observe the heating effect. For hyperthermia treatment of cells, the radiation may be pulsed or may be constant.

The efficiency of heating can be enhanced by an increase of light absorption using doubly doped nanoparticles due to sensitization of multiphonon relaxation as a result of nonradiative energy transfer, for example, from the Tm³⁺ ion in the x % Tm³⁺: y %:Dy³⁺ YPO₄ nanoparticles excited into the ³H₄ level with high absorption cross-section at the ³H₆-³H₄ transition to the Dy³⁺ ion with much weaker the ³H_{15/2}-⁶F_{5/2} absorption transition (FIG. 1).

By selecting the desired concentrations of Tm³⁺ and Dy³⁺ using one type of co-doped nanocrystalline host, e.g. YPO₄, it would be possible to realize simultaneously the laser fluorescent imaging and cancer tumors treatment by the hyperthermia. The ³H₄-³H₆ transition of Tm³⁺ would be used for the fluorescent imaging and the multiphonon transitions of Dy³⁺ for the heating. In this case the submillisecond lifetime of the ³H₄ level allows separate in time the scattering light of the laser and the fluorescence signal using simple and low cost detection system for high contrast of image that is a real advantage over gold nanoparticles with the duration of luminescent signal comparable with that of laser pulse.

By co-doping with different rare-earth ions the nanoparticles can be developed in such a way that they would be used for fluorescent cancer tumour imaging, diagnostics, and local hyperthermia for treatment simultaneously. It is necessary to use different rare-earth ions for imaging and heating. For imaging a low dopant concentration, not higher than 1 mol. %, is required, of a rare earth dopant, for example Nd³⁺ because self-quenching occurs at higher concentrations. For heating the preferable concentrations of rare earth dopants are 30-100%. Therefore, to combine imaging and heating capability in the same nanoparticle a core-shell structured nanoparticle is used with core lightly doped (<1 mol %) for imaging, for example by Nd3+ or Tm3+, and shell heavily doped (30-100 mol %) for heating, for example by Dy3+, otherwise fluorescence quenching of fluorescent dopant (Nd3+) by heating dopant (Dy3+) may occurs.

In general there is no direct limitation on the dimensions of the core and shell apart from the minimal core size, which is possible to synthesize. The shell should not be too thick compared to the core, because independent nanoparticles of shell material are likely to form instead of a shell on the core particle. The following ranges are preferable:

For a core nanoparticle radius in the range of 5 to 50 nm, a shell thickness in the range of 2 to 40 nm is suitable. In general shell thickness is ½ to ⅔ of nanoparticle radius, though it can exceed these limits in some cases.

It is possible for the core host material and shell host material to be the same or different and it is possible for the core dopant and the shell dopant to be the same or different. Some combinations of rare earth dopant for the shell and core are not suitable, for example, Dy³⁺ quenches luminescence of Nd³⁺, Tm³⁺ etc.

To make core-shell nanoparticles, the synthetic procedure is the same, but it becomes two-step. The first step is the preparation of core particles, which is the same as the procedure outlined above to prepare a colloidal solution of the doped nanoparticles. The next step is the synthesis of the shell. This step is also very similar, but to prepare Solution 2 a colloid solution of prepared core particles is used instead of pure water/water:solvent mixture. In this case a gel of shell material is precipitated on the surface of core nanoparticles and crystallizes into a shell during (microwave-) hydrothermal treatment.

The optical material is composed of a dielectric or semiconductor nanoparticles from 2 to 500 nm in size without any conducting material doped solely by Dy³⁺ ions. Alternatively the nanoparticles may be doped solely by Pr³⁺, Nd³⁺, Sm³⁺, Eu³⁺, Tb³⁺, Ho³⁺, Er³⁺, or Tm³⁺ ions or double or triple doped by any combinations of Dy³⁺, Pr³⁺, Nd³⁺, Sm³⁺, Eu³⁺, Tb³⁺, Ho³⁺, Er³⁺, Tm³⁺, and Yb³⁺ ions, as well as any of the colloidal solutions of the nanoparticles, characterized in that the tunable laser radiation with variable output power at selected wavelength is directly transformed inside the material into the energy of crystal lattice vibrations (optical phonons), called the process of multiphonon relaxation, that produces a heat emission from the nanoparticles, which is strong enough to raise the local temperature within several seconds above 45 C and allows smooth variation for degradation of the surrounding biological molecules, organelles, microvessels, and membranes, the excitation wavelength can be in the ultra-violet, visible, near or mid-infrared ranges of the optical spectrum, for either direct excitation of specific rare-earth ions, e.g. Dy³⁺, Pr³⁺, Nd³⁺, Sm³⁺, Eu³⁺, Tb³⁺, Ho³⁺, Er³⁺, Tm³⁺, or using different wavelengths for sensitization of multiphonon relaxation due to nonradiative energy transfer from another excited rare-earth ion with strong absorption transition to that with weaker absorption transition, but with stronger multiphonon transitions.

In the preferred embodiment the dielectric or semiconductor nanoparticle without any conducting material is doped solely by Dy³⁺ ions. Alternatively the nanoparticles may be doped solely by Pr³⁺, Nd³⁺, Sm³⁺, Eu³⁺, Tb³⁺, Ho³⁺, Er³⁺, or Tm³⁺ ions or double or triple doped by any combinations of Dy³⁺, Pr³⁺, Nd³⁺, Sm³⁺, Eu³⁺, Tb³⁺, Ho³⁺, Er³⁺, Tm³⁺, and Yb³⁺ ions.

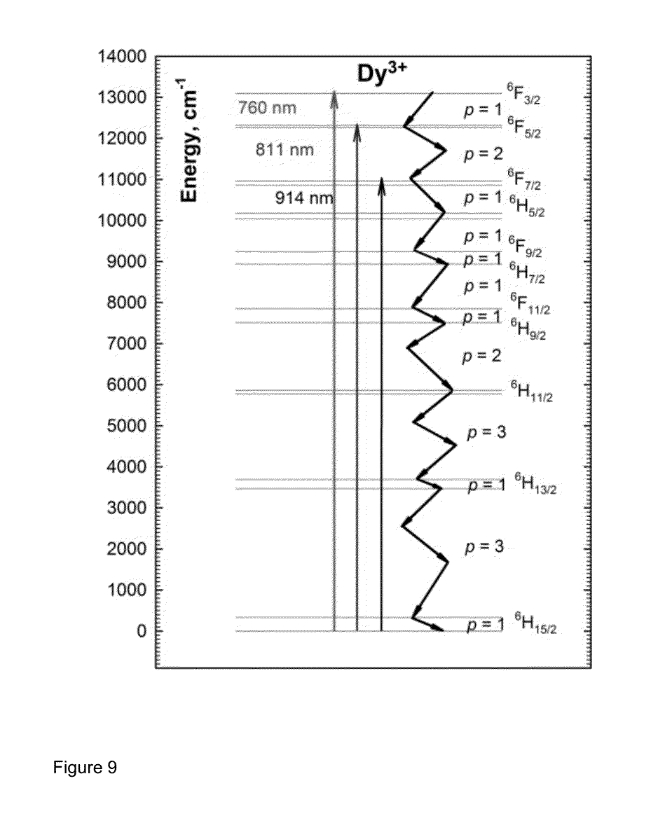

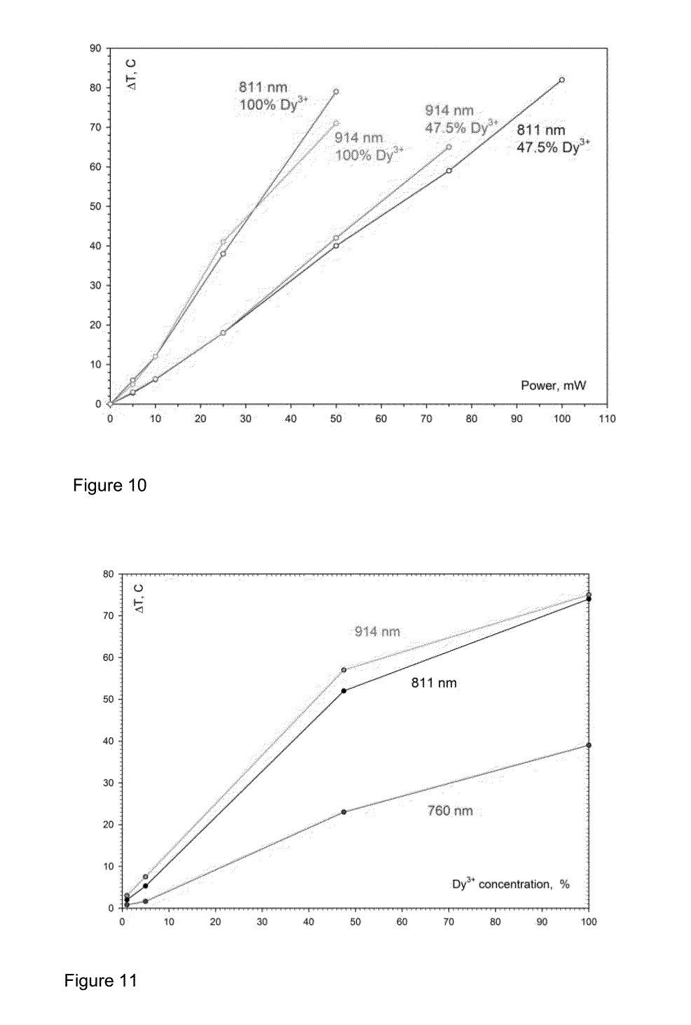

YPO₄ crystalline nanoparticles of 60 nm average size were doped by different concentrations of the Dy³⁺ ion (1, 5, 47.5, and 100%) and excite them directly into the ⁶F_{3/2}, ⁶F_{5/2} (FIG. 1), and ⁶F_{7/2} levels by pulsed tunable laser with 140 fs pulse duration, repetition frequency 80 MHz, and average power up to 140 mW at 760, 811, and 914 nm wavelengths that is into maxima of the spectral peaks of the ⁶H_{15/2}-⁶F_{5/2}; ⁶F_{7/2}; ⁶F_{9/2} optical transitions (FIG. 7) lying in the transparency window of biological tissues. The temperature of the nanoparticles was measured by Remote High Sensitive IR camera JADE MWIR SC7300M, Cedip with the rate of 200 frames per second. As a result a linear increase of the local temperature ΔT with the laser power directly exciting into the ⁶F_{5/2} level of Dy³⁺ in the DyPO₄ nanocrystals was found (FIG. 8) and its linear increase with concentration of the rare-earth dopant in the range from 1 to 47.5 mol. % of Dy³⁺ (FIG. 9). The former is consistent with the following equations for a kinetics of heating derived in the assumption that the thermal equilibrium in the phonon subsystem is set during the time τ much shorter than the flow time of radiative and non-radiative transitions, i.e. the lifetime of nonequilibrium phonons much shorter than the flow times of radiative and non-radiative transitions:

dT/dt=a−b(T−θ) (1)

The first term describes increasing of temperature due to absorbing laser emission and transforming it into heat. The second term describes loss of heat because of transfer of heat energy to environment medium. Newton's law of cooling is used to obtain this term. Here θ is a temperature of environment medium.

a=(N/C_{V})(1−η_{f})(τₚ/τ₀)(σₙₐₙₒ/σ_{bulk})∫σ_{if}(ω)I(ω)dω, (2)

where I(ω)—spectral density of intensity of laser radiation; σ_{if}(ω) is an absorption cross-section of transition from an initial electronic state |i> to an excited state |f> of the bulk crystal with the same crystal matrix as the nanocrystal; N is a number of rare-earth ions in the unit of the volume; C_{V} is a heat capacity at constant volume per unit volume; η_{f} is a fluorescence quantum yield; τₚ is a laser pulse width; τ₀ is a repetition period of the pulses; σₙₐₙₒ/σ_{bulk} is a factor taking into account the difference in the cross sections of nanocrystal and bulk crystal of the same compound;

b=Sh/VC_{V} (3)

and S and V are a surface area and a volume of NP, respectively; h is a heat transfer coefficient. As a result we derived the equation for ΔT(t)=T−Θ as

ΔT(t)=a[|−exp(−bt)]/b. (4)

According to Eq. (3) the loss of heat of the nanoparticle, i.e. transfer of heat to the surrounding medium, is higher for smaller nanoparticles, due to higher the S/V ratio. At the same time transformation of light to heat does not depend on the size (eq. (2)). Therefore the surrounding medium may heat more for smaller nanoparticles, though the temperature of nanoparticles itself is lower.

At the same time the response time of the system is very fast. For example, the rise time of the sample temperature from 33.0 degree° C. to 45.0 degree° C. is approximately one second only and the decay time back to 32.5 degree° C. is approximately 8 seconds (FIG. 6). This indicates very high locality of heating that is important for selective treatment of cancer tumors without disturbing healthy tissues. The decrease from the treatment temperature 45.5 degree ° C. to the pretreatment temperature 41 degree ° C. and back is within one second showing that the surrounding medium barely heats up. Besides, the laser power used for heating is low, 15.5 mW that together with fast heating time means extremely low doses of laser irradiation of the human body for treatment.

We measure a heat efficiency as a ratio of temperature increase to the product of the oscillator strength of absorption transition and laser power value.

η=ΔT/(Pf), (4)

The highest heating efficiency is obtained when exciting into the highest among low lying energy levels of Dy³⁺, more than one degree per mW, and the lowest, 0.3 degree per mW, for excitation into long wavelength energy peak of the near IR spectral band. The heating efficiency does not correlate with the energy of the first multiphonon transition, because an excitation into the middle peak directly into the second top level, which relaxes with 2-phonon transition is higher than to the third one relaxing by one transition.

| | | | | | | | | Number of |
|---|---|---|---|---|---|---|---|---|
| MR | | | | | η = | | | cascade |
| transition | Exc. | | | | ΔT/(Pf), | ΔE, | p, number | processes |
| in DyPO₄ | wavelength | ΔT, C. | P, mW | f, 10⁶ | [C./mW] | cm⁻¹ | of phonons | with p ≦ 3 |
| ⁶F_{3/2}-⁶F_{5/2} | 760 nm | 44 | 110 | 0.32 | 1.25 | 816 | 1 | 12 |
| ⁶F_{5/2}-⁶F_{7/2} | 811 nm | 83.8 | 98 | 1.35 | 0.63 | 1232 | 2 | 11 |
| ⁶F_{7/2}-⁶H_{5/2} | 914 nm | 38 | 39.6 | 3.3 | 0.29 | 713 | 1 | 10 |

The heating efficiency η=ΔT/(Pf) in the DyPO₄ nanocrystals depending on the number of multiphonon transitions with p≦3 in the cascade process. We choose an ion, which has multistage multiphonon relaxation process with single transitions having p≦3 down to ground level in order to exclude loses for photon emission in specific crystal matrix.

Nanoparticles can be delivered to tumor tissue in a number of ways. The first of them is to simply introduce the nanoparticles into the organism; since

the pore diameter of the capillaries of normal tissue is 2-6 nm and in tumor tissue it varies from 2 to 500 nm, nanoparticles larger than 6 nm are more likely to get into the tumor tissue compared with normal tissue. This method can pose problems for organs such as liver, spleen, kidneys or lungs because they also have sufficiently large pore size of blood capillaries and, therefore, tend also to accumulate large nanoparticles. This means that the nanoparticles can pass through these organs without accumulating.

Alternative methods for holding nanoparticles in tumor tissue include:

Chemical delivery. The surface of the nanoparticles can be conjugated with various molecules that have an affinity to various tissues of the tumor or tumor cell organelles.

Biological delivery. To the surface of the nanoparticles are attached antibodies having an affinity to a specific tumor type or tumor specific to a particular patient. This procedure is usually performed immediately prior to administration. Alternatively liposomal formulations can be prepared whereby the nanoparticles are located inside the liposome.

Physical delivery. Nanoparticles can be delivered locally at the tumor producing little physical impact and kept at the tumor site by laser irradiation, ultrasonic manipulation or magnetic confinement.

Nanoparticles of the same size are preferable used. However, for some cases it is advantageous to use multiple sizes. This way we can more accurately determine the prevalence of the tumor as nanoparticles of different sizes will be accumulated in different parts tumor tissue.

The functionalised nanoparticles (i.e nanoparticles conjugated with antibodies or included in a liposomic structure) can then be combined in a pharmaceutical composition such as saline solution ready for administration to a human, animal or in vitro cells.

The nanoparticles can be used for in vivo or vitro treatment of cells where there is over proliferation of cells, particularly malignant cell, but also non-malignant cell and cosmetic removal of cells. The idea of heating is centered on the process of multiphonon relaxation (MR) of the optical excitation energy in the RE doped crystals. In the field of rare-earth doped fluorescent and laser materials intra-center multiphonon relaxation usually competes with radiative relaxation. In the single frequency model of lattice vibrations a decrease of the phonon number p=ΔE/Øω_{eff.} bridging the energy gap ΔE between two electronic levels by one raises the rate of multiphonon transition by one or two orders of magnitude. If the number of phonons is equal or less than three (p≦3), the RE ions fluorescence almost completely quenches by multiphonon relaxation, because the rate of multiphonon transition is on the nanosecond or even picosecond time scale that is 10⁵-10⁷ times faster than the spontaneous emission decay rate of the RE ions.

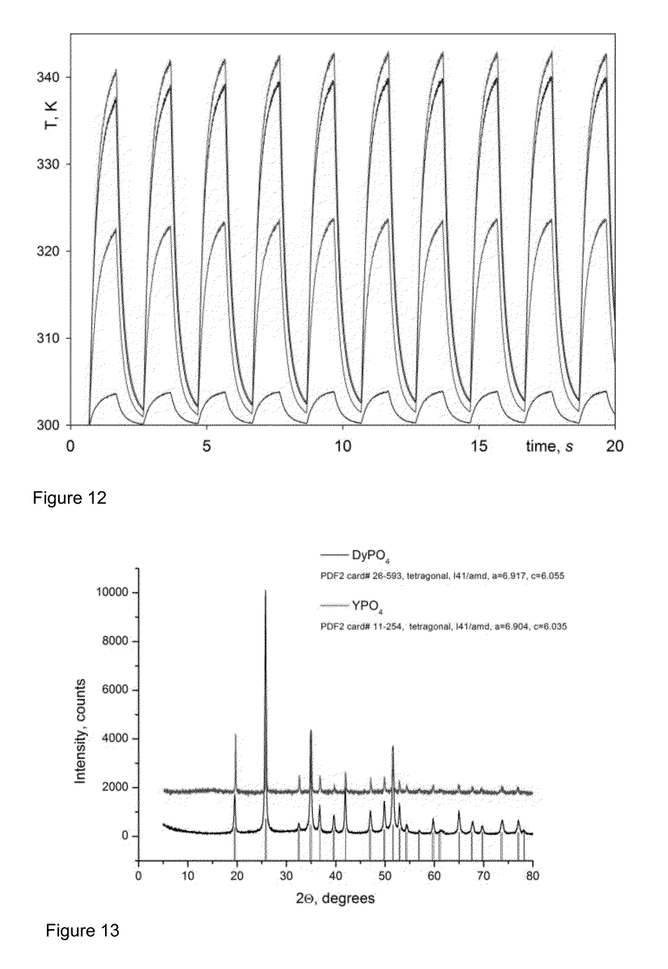

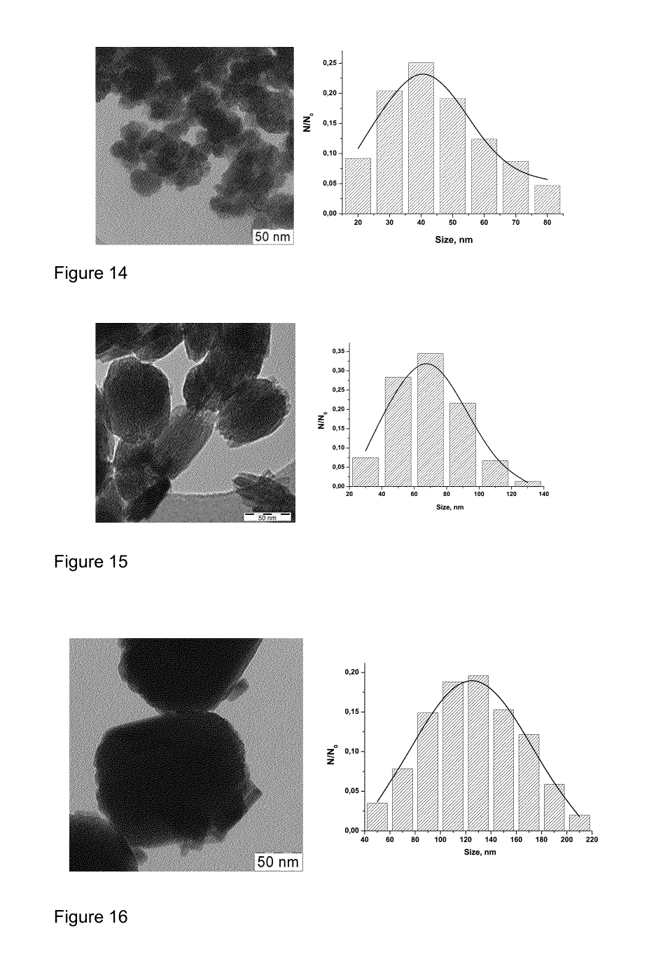

However, the negative effect of MR in case of fluorescent materials can be used as a positive effect for nanoscaled heaters. We propose “non-fluorescent” nanocrystals instead of fluorescent ones. For this we reduce a fluorescent quantum yield from almost unity typical for metastable levels of the RE ions to 10⁻⁵-10⁻⁷ raising the rate of MR comparing to radiative rate. In so doing we choose the RE ion, which enables immediately after laser irradiation to start multistage (cascade) multiphonon relaxation process down to the ground level with efficiency of photon energy transformation to heat close to unity. A Dy³⁺ ion embedded into the YPO₄ crystal matrix having simultaneously a wide phonon spectrum (Øω_{max.}=1100 cm⁻¹) and permitting up to 100% substitution of the Dy³⁺ ion for Y³⁺ meets this requirement. The energy level diagram of the Dy³⁺ ion allows for choosing laser excitation wavelength between 760 (⁶F_{3/2}), 811 (⁶F_{5/2}), or 914 nm (⁶F_{7/2}) FIGS. 11, 12 in the near IR spectral range fitting the transparency window of biological tissues (750-950 nm). It has real advantage over the fluoride crystal matrixes, where even in the LiYF₄ host crystal with the most extended phonon spectrum among fluoride crystals the maximal phonon energy does not exceed Øωₘₐₓ=560 cm⁻¹. The use of the fluoride hosts would increase the number of phonons bridging the energy gaps for the ⁶H_{11/2}→⁶H_{13/2} and ⁶H_{13/2}→⁶H_{15/2} transitions of Dy³⁺ to four and five for LiYF₄ and to six and seven for the LaF₃ crystal host, respectively. The latter has the lowest maximal phonon frequency Øω_{max.}=400 cm⁻¹ among fluoride crystals. As a result the MR rates of the ⁶H_{11/2} and ⁶H_{13/2} levels in the Dy³⁺ doped fluoride matrixes would have been comparable with their spontaneous emission decay rates, and significant amount of the optical excitation energy would have emitted by photons rather than phonons. However, for highly concentrated Dy³⁺ doped fluoride samples like DyF₃ the concentration quenching may compensate the low phonon spectrum of the matrix.

The temperature of NP increases linearly with increasing the spectral density of absorbed laser radiation and concentration of RE dopant (Eqs. (1-4).

Laser heating by 811 and 914 nm wavelengths of samples prepared using microwave-hydrothermal treatment of freshly precipitated gels demonstrates almost linear dependence of the local temperature increase ΔT on the laser power of a powder surface hottest area (425×425 μm) taking from the central pixel of the camera image for direct excitation into the ⁶F_{5/2} and ⁶F_{7/2} levels of Dy³⁺ in the DyPO₄ and Y_{0.525}Dy_{0.475}PO₄ nanocrystals as shown in FIG. 13. Also, the heating increases in proportion with increasing of Dy³⁺ concentration.

Also a linear increase of the local temperature increase ΔT of the powder hottest area with the increase in Dy³⁺ concentration in the range from 1 to 47.5 mol. % of Dy³⁺ and low decline from linearity for DyPO₄ for all three excitation wavelengths, 760, 811, and 914 nm FIG. 4, was found. This latter may be attributed to surface water evaporation.

The heating efficiency of the powder Φ=ΔT/(Pf) was measured as a ratio of its local temperature increase (ΔT) to the product of the oscillator strength of the absorption transition (f) and the quantity of laser power (P). We obtained the highest heating efficiency, more than one degree per mW when exciting into the top ⁶F_{3/2} level of Dy³⁺ in the DyPO₄ nanocrystals, and the lowest, less than 0.3 degree per mW, while exciting into the third top the ⁶F_{7/2} level, which both relax by one-phonon transition. Also, we excited the second top the ⁶F_{5/2} level, which relaxes by 2-phonon transition, and obtained higher heating efficiency than exciting into the third top the ⁶F_{7/2} level. We found that the heating efficiency does not correlate with the MR rate of the excited level. Otherwise it would be higher for the ⁶F_{7/2} level relaxing with the emission of just one phonon than for ⁶F_{5/2} relaxing with the emission of two phonons. We conclude that in the system under study the efficiency of heating is proportional to a number of multiphonon transitions with p≦3 in the cascade nonradiative relaxation process, which is maximal for the upper ⁶F_{3/2} level (N=12) and minimal for the lowest ⁶F_{7/2} level (N=10), or in other words the higher is the initially excited level, the more electronic energy is transferred to heat, ceteris paribus. A decrease of the Dy³⁺ concentration reduces the heating efficiency in accordance with the dependence shown in FIG. 13.

At the same time, the heating time of the powder is very short. For example, the rise time of the temperature from room temperature to 340K at the hottest spot of the DyPO₄ powder under pulsed 811 nm laser irradiation in the scanning microscope spot mode with average power of 30 mW is approximately one second only, see FIG. 15, upper curve. The temperature drops back to room temperature within the same time. This is an indication of inertialess heating, which enables setting precise duration for hyperthermia, which may be very important for selective treatment of cancer tumors without disturbing healthy tissues. Besides, the laser power used for heating was low, tens of mW only that together with fast heating time to the required temperature may result in low doses of laser irradiation of human body during hyperthermia treatment. We found that the rates of heating and cooling are independent of excitation wavelength FIG. 15. The laser irradiation at 850 nm, which is out of energy resonance with Dy³⁺ energy levels FIG. 11, does not indicate significant heating of the powder FIG. 15, lower curve, which confirms that the heating is a result of the multiphonon relaxation from the excited Dy³⁺ electronic states.

It is seen that the temperature of the NPs can be rather high, much higher than it is necessary for cancer hyperthermia treatment. However for colloidal solutions the maximal achievable local temperatures will be lower than for the powders. This requires a separate study including the development of direct and indirect methods for the temperature measurements.

It is possible to synthesize the core-shell NPs with photon emissive core doped by Nd³⁺ ion and heat emissive shell doped by Dy³⁺ using the same YPO₄ crystal matrix for simultaneous near IR tumor imaging and cancer laser hyperthermia treatment.

### Further Example Sample Preparation

As starting compounds for preparation of the Y₁₋ₓDyₓPO₄ nanoparticles (x=0.01, 0.05, 0.475 and 1) we used DyCl₃.6H₂O (Aldrich, 99.995% purity), Y(NO₃)₃.4H₂O (Aldrich, 99.999% purity) and K₂HPO₄.3H₂O (Aldrich, 99.9% purity). For the synthesis we prepared solutions of 5 mmols of the mixture of DyCl₃.6H₂O and Y(NO₃)₃.4H₂O, taken in corresponding stoichiometric proportions, in 10 ml of deionized water, as well as solution of 5 mmols of K₂HPO₄.3H₂O in 30 ml of deionized water. After that we added the solution of rare-earth salts drop-wise to the solution of phosphate under vigorous stirring and left it for 15 min keeping the stirring on. We diluted the freshly precipitated gel in mother solution with 10 ml of deionized water, transferred it into 100 ml Teflon autoclave and expose to microwave-hydrothermal (MW-HT) treatment (200° C., 2 hours) using a Berghof Speedwave-4 laboratory device (2.45 GHz, 1 kW maximum output power). After the treatment the samples were centrifuged, washed several times with deionized water and air-dried at 200° C. for 2 hours.

XRD analysis of obtained samples (NPs powder) shown in FIG. 16 that they consist of pure tetragonal phase with I4₁/amd space group, isostructural to xenotime YPO₄. It is worthy to note that isostructural YPO₄ and DyPO₄ phases have almost equal lattice parameters, and due to lanthanide contraction the radius of the Dy³⁺ ion is very close to the one of Y³⁺ ion. Therefore, regardless of the Y:Dy ratio in solid solution, the positions of maxima on XRD patterns remain almost the same. The synthesized samples of Y₁₋ₓDyₓPO₄ possess high degree of crystallinity. The mean size of the coherent scattering region (CSR) depends on the value of x and changes from 40±3 nm for x=0.01 to around 150 nm for x=1 (the precision of CSR size determination for large sizes is rather low due to small physical broadening).

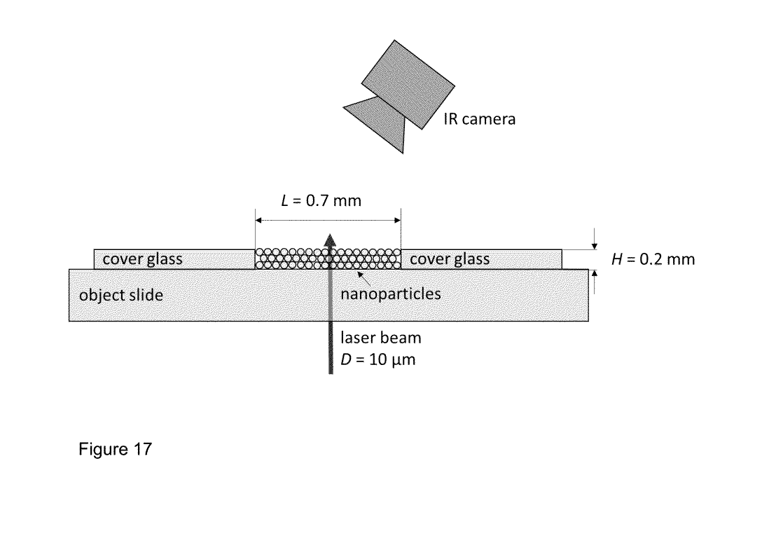

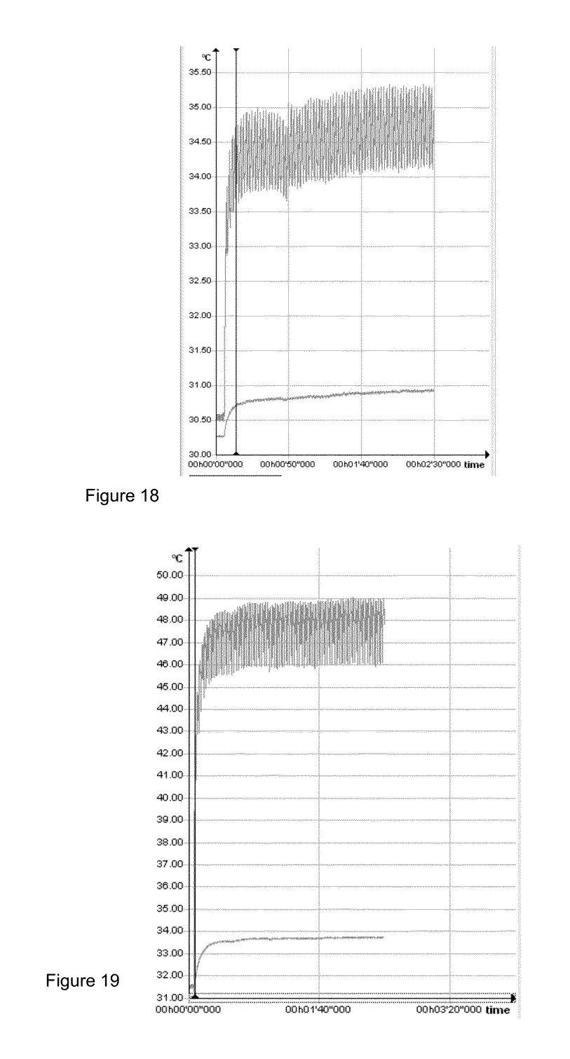

Morphology of the synthesized nanoparticles was studied by means of TEM shown in FIGS. 17 to 19. Nanoparticles are isotropic and rather uniform. Mean size of the particles determined using the TEM data is 40±15 nm for x=0.01, 65±22 nm for x=0.475, and 125±45 nm for x=1, which is in good correlation with XRD results and confirms the high degree of crystallinity of all the samples. The width of the size distribution does not vary substantially for synthesized samples. It is close to normal with slight admixture of lognormal component, which decreases with the decrease in the dysprosium content. For nanoparticles of pure DyPO₄ one can see in FIG. 19 that apart from the main fraction of cuboid shape large particles, some amount of significantly smaller rod-like nanoparticles is present. Closer look to the inner structure of the larger particles and the remaining aggregates of the smaller nanoparticles allows one to suggest that formation of larger nanoparticles is due to oriented attachment and growth of rod-like nanoparticles, rather than due to Ostwald ripening. The same most likely applies to the Y_{0.525}Dy_{0.475}PO₄ nanoparticles. As for the Y_{0.99}Dy_{0.01}PO₄ nanoparticles it is hard to deduce from TEM micrographs whether they formed mainly by aggregation or growth.

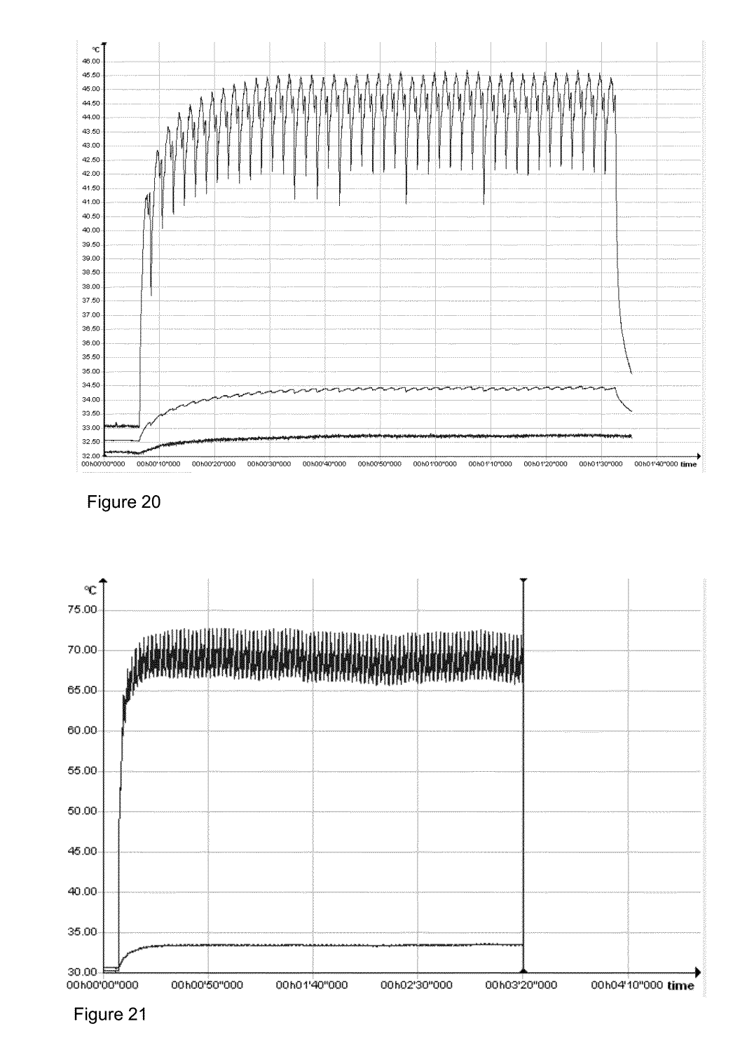

Measurements quoted above were obtained as follows: Dy³⁺ ions were excited directly into the ⁶F_{3/2}, ⁶F_{5/2}, or ⁶F_{7/2} levels by pulsed tunable femtosecond laser Coherent Chameleon Ultra II with 140 fs pulse duration, repetition frequency 80 MHz, and maximal average power up to 140 mW. We used 760, 811 and 914 nm wavelengths that is into the maxima of the spectral peaks of the ⁶H_{15/2}→⁶F_{3/2}, ⁶F_{5/2}, and ⁶F_{7/2} optical transitions of Dy³⁺, respectively lying in the transparency window of biological tissues (750-950 nm). Approximately 65 mg of the nanoparticles powder was poured between two cover glasses with a width of 1.0 mm and a thickness of 0.2 mm, laid on a glass slide. The distance between the cover glasses is 0.7 mm. So, the volume of the sample was 0.14 10⁻³ cm³ in FIG. 20. The excess of the powder (powder protruding above the surface of the cover glass) has been removed from the surface. The laser beam was focused on the bottom surface of the powder layer through the slide. Temperature readout was taken from the upper layer of powder. The laser irradiation was done through scanning microscope ZEISS LSM 710 in a spot mode. The laser spot had the diameter of 10 μm. The ellipticity of the laser beam is 0.9-1.1. We measured the temperature of nanoparticles by Remote High Sensitive IR camera JADE MWIR SC7300M, Cedip sensitive in the mid IR (3-5 μm) spectral range with maximal time resolution 6.7 ms. The size of a single camera pixel was 530×530 μm, which is a limit for spatial resolution of the temperature measurements. The image of the area in the spot mode was projected onto the four pixels of the IR camera.

While the invention has been described with reference to the embodiments above, a person of ordinary skill in the art would understand that various changes or modifications may be made thereto without departing from the scope of the claims.

We claim:

1. A collection of nanoparticles suitable for use in hyperthermia treatment by light irradiation at a wavelength in the transparency window of biological tissue, wherein each nanoparticle comprises:
a crystalline host structure, and
at least one species of rare-earth dopant ion.

2. A collection of nanoparticles in accordance with claim 1, wherein the concentration of the at least one species of dopant is in the range 30 to 100 molecular %.

3. A collection of nanoparticles in accordance with claim 1, wherein the concentration of the at least one species of dopant is in the range 80 to 100 molecular %.

4. A collection of nanoparticles in accordance with claim 1, wherein the concentration of the at least one species of dopant is in the range 90 to 100 molecular %.

5. A collection of nanoparticles in accordance with claim 1, wherein the concentration of the at least one species of dopant is in the range 95.0 to 100.0 molecular %.

6. A collection of nanoparticles in accordance with claim 1, wherein the at least one species of rare-earth dopant ion is selected from the list of Dy³⁺, Pr³⁺, Nd³⁺, Sm³⁺, Eu³⁺, Tb³⁺, Ho³⁺, Er³⁺, and Tm³⁺ ions.

7. A collection of nanoparticles in accordance with claim 1, wherein the crystalline host structure is a dielectric material.

8. A collection of nanoparticles in accordance with claim 7, wherein the dielectric is a phosphate, a vanadate, a molibdate, a tungstate, an oxide or a fluoride.

9. A collection of nanoparticles in accordance with claim 1, wherein the crystalline host structure is a semiconductor material.

10. A collection of nanoparticles in accordance with claim 1, wherein the average diameter of the nanoparticles is in the range 5 to 500 nm

11. A collection of nanoparticles in accordance with claim 1, wherein the average diameter of the nanoparticles is in the range 20 to 60 nm.

12. A collection of nanoparticles in accordance with claim 1, wherein the crystalline host structure may be double or triple doped by any combinations of Dy³⁺, Pr³⁺, Nd³⁺, Sm³⁺, Eu³⁺, Tb³⁺, Ho³⁺, Er³⁺, Tm³⁺, and Yb³⁺ ions.

13. A collection of nanoparticles in accordance with claim 1, wherein the nanoparticles have a core of a first host material doped with one or more types of rare earth dopant ions and a shell of a second host material doped with one or more types of rare earth dopant ions.

14. A collection of nanoparticles in accordance with claim 13, wherein the concentration of the dopant ions in the core is less than 1 mol % and the concentration of the dopant ions in the shell is in the range 30-100 mol %.

15. A collection of nanoparticles in accordance with claim 1, wherein the nanoparticles are conjugated with molecules that specifically bind to a target cell.

16. A collection of nanoparticles in accordance with claim 15, wherein the conjugated molecules are antibodies suitable for the formation of an antigen/antibody complex with the target cell.

17. A collection of nanoparticles in accordance with claim 15, wherein the conjugated molecules are liposomes having targeting ligands suitable for the formation of a ligand/receptor complex with the target cell.

18. A collection of nanoparticles in accordance with claim 1 for use in the hyperthermia treatment of over-proliferating cells.

19. A collection of nanoparticles in accordance with claim 1 for use in the hyperthermia treatment of over-proliferating cells, wherein the over-proliferating cells are malignant.

20. A pharmaceutical composition containing the collection of nanoparticles of claim 1.

21. A method of inducing localised hyperthermia in target cells comprising the steps of delivering nanoparticles of the type claimed in claim 1 to cells and exposing the nanoparticles to electromagnetic radiation.

22. A method of inducing localised hyperthermia and imaging target cells, comprising the steps of delivering nanoparticles of the type claimed in claim 1 to cells and exposing the nanoparticles to electromagnetic radiation and detecting the absorption, fluorescence or scattering of the radiation to simultaneously heat and view the target cells.

23. A method of inducing localised hyperthermia in target cells in accordance with claim 21, wherein the electromagnetic radiation has a wavelength in a biological transparency window of 800-900 nm.

24. A method of inducing localised hyperthermia in target cells in accordance with claim 21, wherein the method is applied to cells in vitro.

25. A method of inducing localised hyperthermia in target cells in accordance with claim 21, wherein the conjugated molecules are antibodies suitable for the formation of an antigen/antibody complex with the target cell.
